(19) United States
(12) Patent Application Publication
(10) Pub. No.: US 2007/0031826 A1
(43) Pub. Date: Feb. 8, 2007

(54) Diagnostic kit for determining the genotype of a human papilloma virus and method of using thereof

(75) Inventors: Duck-Joo Lee, Holladay, UT (US); Woon-Won Jung, Danjee (KR); Hyun-Sook Kim, Seoul (KR); Inkwon Han, Hyundae Litzville (KR)

Correspondence Address: THORPE NORTH & WESTERN, LLP., 8180 SOUTH 700 EAST, SUITE 200, SANDY, UT, 84070, US

(73) Assignee: MY GENE, Seoul (KR)

(21) Appl. No.: 11/497,184

(22) Filed: Jul. 31, 2006

Related U.S. Application Data
(60) Provisional application No. 60/705,792, filed on Aug. 5, 2005.

(51) Int. Cl. C12Q 1/70; G01N 1/28; C12Q 1/68; C12M 1/34

(52) U.S. Cl. 435/5; 435/6; 435/287.2; 427/2.11; 977/924

(57) ABSTRACT

This invention relates to a kit and a method of use thereof to detect and determine the genotype of HPVs in a biological sample. The kit for detection and determination of the genotype of HPV, comprises PCR primers for HPV genes; HPV oligonucleotide chips having one or more probes immobilized on the chip which have nucleotide sequences complementary to the DNA of 24 HPV subtypes; means and regents for collecting, extracting and amplifying the DNA from a biological sample and hybridizing the amplified DNA with the HPV oligonucleotide chip; and instructions for using the kit to detect and determine the HPV genotype present in the sample.

### CROSS-REFERENCE TO RELATED APPLICATIONS AND CLAIM OF PRIORITY

Priority of U.S. provisional patent application Ser. No. 60/705,792 filed on Aug. 5, 2005 is claimed.

### BACKGROUND

The present invention relates generally to a diagnostic kit and method of using thereof for determining the genotype of HPV DNA in cervical samples. More particularly, the present invention relates to a method of determining the genotype of HPV DNA in cervical samples using a PCR method and oligonucleotide chip. The present invention provides a method for determining the genotype of HPV DNA present in samples collected during cervical cancer screening in patients infected with HPV.

Cervical cancer is the second most common cancer in terms of both incidence and mortality worldwide. To date, there seems to be a limitation to cytology-based screening programs in terms of reducing cervical carcinoma incidence and mortality rates. Recently, it has been recognized that the availability of highly sensitive human papillomavirus (HPV) tests offers the potential for replacement of conventional cytologic screening programs. In addition, the importance of HPV testing is increased because genetic typing is required to predict clinical progression.

Cervical cancer has usually been detected by examining cervical PAP smear samples for dysplasia under a microscope. However, since this relies on visual examination, false-positive and false-negative results appear frequently. In addition, the PAP smear method requires highly trained experts who can differentiate cellular abnormalities. Therefore, a method to more accurately and easily detect cervical cancer is needed. Recently, it has been reported that the HPV DNA test is more effective than the PAP smear test from studies that have shown that the human Papilloma virus (HPV) causes cervical cancer.

HPV is a double-stranded circular DNA virus that has about 8,000 base pairs. About 80 different genotypes of HPV are known to date and they can be divided into a low-risk (HPV-6, -11, -34, -40, -42, -43, -44, -70) and a high-risk group of genotypes (HPV-16, -18, -31, -33, -35, -39, -45, -51, -52, -53, -54, -56, -58, -59, -66, -68). More than 35 distinctive HPV genotypes have been associated with cervical epithelial neoplasia (CIN) and cervical cancer. But, the traditional methods for HPV detection, such as morphological and immunological methods, show low sensitivity and specificity, and do not detect the specific HPV genotype present. A new HPV detection technique using a DNA microarray (DNA chip) has recently been developed. This technique is based on the polymerase chain reaction (PCR) method and has the advantage of high sensitivity and the ability to detect single and multiple infections at same time. However, these methods have many disadvantages, such as requiring a great deal of time and labor to perform the test, the risk of using radioactive isotopes and cancer-causing DNA staining reagents, and inconvenience in the processes of sample management. Therefore, these methods are unsuitable for daily use in clinical settings.

### SUMMARY

It has been recognized that it would be advantageous to develop a DNA chip for easy, sensitive, and accurate determination of HPV genotypes, which subsequently can be used clinically for cervical cancer screening. To solve the above-mentioned problems of traditional HPV testing methods, the present invention provides a device and a method of use thereof that can determine many genotypes of HPV at the same time. More specifically, the present invention provides a HPV genotype analysis kit for analyzing the HPV genotype in a patient infected with HPV by using a DNA chip, and a method for diagnosing HPV infection by analyzing the patient's HPV genotype and predicting risk of cervical cancer.

One embodiment of the present invention relates to a method of determining the genotype of HPVs present, comprising collecting cells from the cervix uteri, extracting DNA from the cells, amplifying the extracted DNA using a two step nested PCR method, hybridizing the amplified HPV DNA on an oligonucleotide chip for the HPV genotype test, and determining the genotype of the specifically bound HPV DNA after hybridization.

Preferably, the reagents used in the DNA extraction process contain a protease. The present invention provides a kit for determining the genotype of HPV, which comprises: a) a tool for collection of cervical cells and reagents for DNA extraction; b) novel reagents for amplification of the HPV DNA by a two step nested PCR method; c) an oligonucleotide chip containing probes specific for certain HPV genotypes, and d) instructions for processing the samples and testing in either a centralized facility using standard equipment or at the point of care (e.g., physician's office) using appropriate testing equipment and rapid sample-handling technology.

### BRIEF DESCRIPTION OF THE DRAWINGS

Additional features and advantages of the invention will be apparent from the detailed description which follows, which when taken in conjunction with the accompanying drawings, together illustrate, by way of example, features of the invention; and, wherein:

FIG. 1 is a schematic outline of the HPV DNA genome;

FIG. 2 illustrates the location of the PCR product using the GP primer and MY primer sets of the present invention;

FIG. 3 illustrates a micro-array pattern of 24 HPV types probes spotted on one of the eight regions on a glass chip;

FIG. 4-8 illustrates the HPV genotyping results of cervical samples using the DNA chips of the present invention.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

Before the present HPV genotyping kit and methods of using and making thereof are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise. Thus, for example, reference to a chip containing “a probe” includes reference to two or more such probes, and reference to “a HPV genotype” includes reference to two or more of such genotypes.

In describing and claiming the present invention, the following terminology will be used in accordance with the definitions set out below.

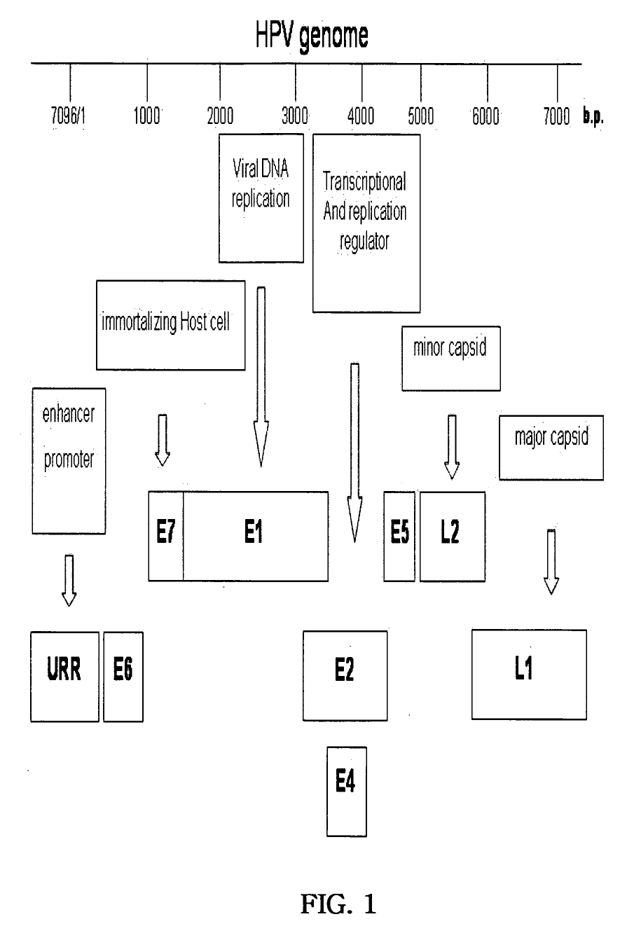

As illustrated in FIG. 1, HPV isolates that display a sequence difference of more than 10% to any previously known type in the combined nucleotide sequences of E6, E7 and L1 genes are classified as different HPV “genotypes.” HPV isolates that differ between 2 and 10% are classified as different “subtypes.” If the sequence variation is below 2%, the isolates are classified within the same subtype as different “variants.” The term “type” when applied to HPV refers to any of the three categories defined above.

The target material in the samples to be analyzed may either be DNA or RNA, e.g. genomic DNA, messenger RNA, viral RNA or amplified versions thereof. These molecules in this application are also termed “polynucleic acids.”

Well-known extraction and purification procedures are available for the isolation of RNA or DNA from a sample.

The term “probe” according to the present invention refers to a single-stranded oligonucleotide which is designed to specifically hybridize to HPV polynucleic acids.

The term “primer” refers to a single stranded oligonucleotide sequence capable of acting as a point of initiation for synthesis of a primer extension product which is complementary to the nucleic acid strand to be copied. The length and the sequence of the primer must be such that they allow the synthesis of the extension products. Preferably the primer is about 5-50 nucleotides long. The specific length and sequence will depend on the complexity of the required DNA or RNA targets, as well as on the conditions under which the primer is used, such as temperature and ionic strength.

The expression “suitable primer pair” in this invention refers to a pair of primers allowing the amplification of part or all of the HPV polynucleic acid fragment on which the probes are immobilized.

The term “target sequence” of a probe or a primer according to the present invention is the sequence within the HPV polynucleic acid to which the probe or the primer is completely complementary or partially complementary (i.e. with some degree of mismatch). It is to be understood that the complement of said target sequence is also a suitable target sequence in some cases. The probes of the present invention should be complementary to at least the central part of their target sequence. In most cases the probes are completely complementary to their target sequence. The term “type-specific target sequence” refers to a target sequence within the polynucleic acids of a given HPV type that contains at least one nucleotide difference as compared to any other HPV-type.

“Specific hybridization” of a probe to a region of the HPV polynucleic acids means that, after the amplification step, said probe forms a duplex with part of this region or with the entire region under the experimental conditions used, and that under those conditions said probe does not form a duplex with other regions of the polynucleic acids present in the sample to be analyzed. It should be understood that probes which are designed for specific hybridization to a region of HPV polynucleic acids may fall within said region or may to a large extent overlap with said region (i.e. form a duplex with nucleotides outside as well as within said region).

“Specific hybridization” of a primer to a region of the HPV polynucleic acids means that, during the amplification step, said primer forms a duplex with part of this region or with the entire region under the experimental conditions used, and that under those conditions said primer does not form a duplex with other regions of the polynucleic acids present in the sample to be analyzed. It should be understood that primers that are designed for specific hybridization to a region of HPV polynucleic acids may fall within said region or may to a large extent overlap with said region (i.e., form a duplex with nucleotides outside as well as within said region).

Since the current application requires the detection of single base pair mismatches, stringent conditions for hybridization of the probes are required, allowing only hybridization of exactly complementary sequences. However, it should be noted that, since the central part of the probe is essential for its hybridization characteristics, possible deviations of the probe sequence versus the target sequence may be allowable towards the extremities of the probe when longer probe sequences are used. Variations are possible in the length of the probes. Said deviations and variations, which may be conceived from common knowledge in the art, should however always be evaluated experimentally in order to check if they result in equivalent hybridization characteristics as the exactly complementary probes.

Preferably, the probes of the invention are about 5 to 50 nucleotides in length, and more preferably are from about 10 to 30 nucleotides in length. The nucleotides as used in the present invention may be ribonucleotides, deoxyribonucleotides and modified nucleotides such as inosine or nucleotides containing modified groups, which do not essentially alter their hybridization characteristics.

Probe sequences are represented throughout the specification as single stranded DNA oligonucleotides from the 5′ to the 3′ end. It will be obvious to one skilled in the art that any of the below-specified probes can be used as such, or in their complementary form, or in their RNA form (wherein T is replaced by U).

The probes according to the present invention can be prepared by cloning of recombinant plasmids containing inserts including the corresponding nucleotide sequences, if need be by excision of the latter from the cloned plasmids by the use of adequate nucleases and recovering them, e.g., by fractionation according to molecular weight. The probes according to the present invention can also be synthesized chemically, for instance by conventional phospho-triester methods.

The oligonucleotides used as primers or probes may also comprise nucleotide analogues such as phosphorothiates, alkylphosphorothiates or peptide nucleic acids or may contain intercalating agents. As with most other variations or modifications introduced into the original DNA sequences of the invention these variations will necessitate adaptations with respect to the conditions under which the oligonucleotide should be used to obtain the required specificity and sensitivity. However the eventual results of hybridization will be essentially the same as those obtained with the unmodified oligonucleotides. The introduction of these modifications may be advantageous in order to positively influence characteristics such as hybridization kinetics, reversibility of the hybrid-formation, biological stability of the oligonucleotide molecules, etc.

The term “solid support” can refer to any substrate to which an oligonucleotide probe can be coupled, provided that it retains its hybridization characteristics and provided that the background level of hybridization remains low. Usually the solid substrate will be a microtiter plate (e.g., in the DEIA technique), a membrane (e.g., nylon or nitrocellulose) a microsphere (bead) or a chip. Prior to application to the membrane or fixation it may be convenient to modify the nucleic acid probe in order to facilitate fixation or improve the hybridization efficiency. Such modifications may encompass homopolymer tailing, coupling with different reactive groups such as aliphatic groups, NH₂ groups, SH groups, carboxylic groups, or coupling with biotin, haptens, or proteins.

The term “labeled” refers to the use of labeled nucleic acids. Labeling may be carried out by the use of labeled nucleotides incorporated during the polymerization step of the amplification process or by use of labeled primers, or by any other method known to the person skilled in the art. The nature of the label may be isotopic or non-isotopic (Cy5 fluorescent, biotin, digoxigenin, etc.).

The “sample” may be any biological material taken either directly from the infected human being (or animal), or after culturing (enrichment). Biological material may be scrapings or biopsies from the urogenital tract or any other part of the human or animal body.

Reference will now be made to the exemplary embodiments and specific language will be used herein to describe the same. It will nevertheless be understood that no limitation of the scope of the invention is thereby intended. Alterations and further modifications of the inventive features illustrated herein, and additional applications of the principles of the invention as illustrated herein, which would occur to one skilled in the relevant art and having possession of this disclosure, are to be considered within the scope of the invention.

One aspect of the present invention provides a kit and a method of determining the genotype of HPV present in the sample, comprising collecting cells from the cervix uteri, extracting DNA from the cells, amplifying the extracted DNA using a two step nested PCR method, hybridizing the amplified HPV DNA on a oligonucleotide chip for the HPV genotype test, and examining the genotype of the specifically bound HPV DNA after hybridization. Preferably, the reagents used in the DNA extraction process contain a protease.

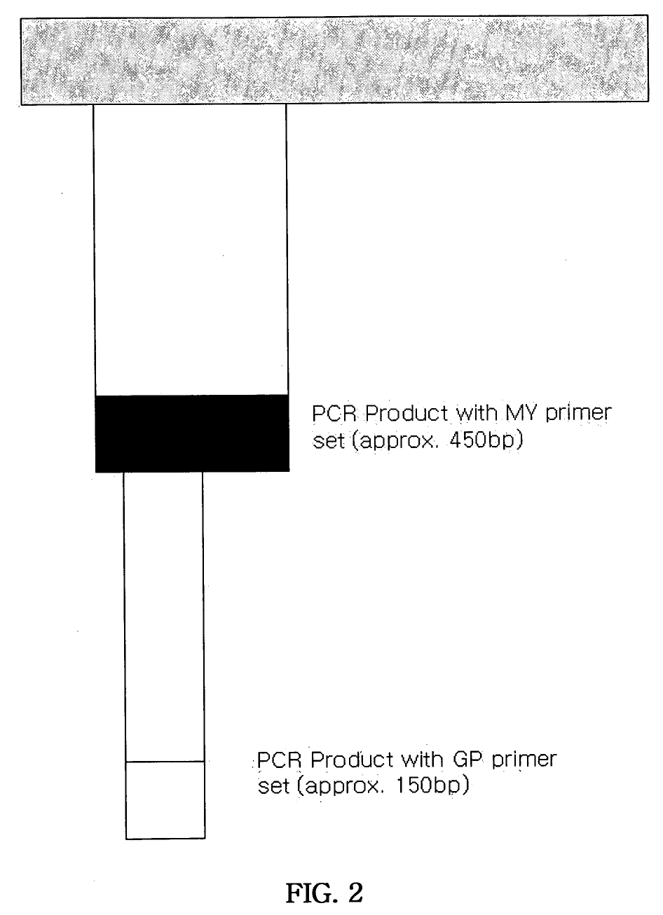

Two step nested PCR provides a powerful method to amplify specific sequences of DNA from a large complex mixture of DNA. One pair of PCR primers can amplify a single locus from an entire genome using a single template molecule, over 1 billion copies of the PCR product can be produced very quickly. However, the capacity to amplify over one billion fold also increases the possibility of amplifying the wrong DNA sequence over one billion times. The specificity of PCR is then determined by the specificity of the PCR primers. For example, if the primers bind to more than one locus (e.g., paralog or common domain), then more than one segment of DNA will be amplified. In order to control for these possibilities, investigators often employ nested primers to ensure specificity. A nested PCR means that two pairs of PCR primers were used for a single locus (FIG. 2). The first pair amplifies the locus as seen in any conventional PCR. The second pair of primers (nested primers) binds within the first PCR product and produces a second PCR product that is shorter than the first one. The rational behind this strategy is that if the wrong locus were to be amplified by mistake, the probability is very low that it would also be amplified a second time by a second pair of primers. Therefore, the two step nested PCR employed in the present invention provides for more specific detection of the HPV genotype present in biological samples. The primers used in the present invention for HPV genes have the sequences depicted in SEQ. ID NO: 1, SEQ. ID NO: 2, SEQ. ID NO: 3 and SEQ. ID NO: 4 (Table 1).

TABLE 1

PCR primer sequence of HPV DNA

| | |
|---|---|
| Name of primer | Primer sequence(5′→3′) |

| | |
|---|---|
| My09 | 5′-CGT-CC(A/C)-A(A/G)(A/G)-GGA- |
| (7032-7014 bp) | (A/T)AC-TGA-TC-3′ |
| (SEQ. ID NO: 1) | |
| MY11 | 5′Cy3-GC(A/C)-CAG-GG(A/T)-CAT- |
| (6582-6602 bp) | AA(C/T)-AAT-GG-3′ |
| (SEQ. ID NO: 2) | |
| GP + 5 | 5′-TTT-GTT-ACT-GTG-GTA-GAT-ACT-AC- |
| (6624-6646 bp) | 3′ |
| (SEQ. ID NO: 3) | |
| Cy5-GP + 6 | 5′Cy5-GAA-AAA-TAA-ACT-GTA-AAT-CAT- |
| (6765-6741 bp) | ATT-C-3′ |
| (SEQ. ID NO: 4) | |

In order to quickly and accurately determine the HPV genotype using the DNA chip of the present invention, twenty four specific probes which are capable of complementarily annealing with 24 types of HPV are immobilized in a micro-array pattern on a chip which can then detect 24 HPV genotypes simultaneously. The inventive probes of the present invention have the nucleotide sequences depicted in (SEQ. ID: 5-SEQ. ID: 28) (Table 2).

TABLE 2

sequence of probes for HPV genotype test
(5′-3′)

| | |
|---|---|
| Probe | Sequence(5′-3′) |

| | |
|---|---|
| HPV6 | ATC CGT AAC TAC ATC TTC CAC ATA |
| (SEQ. ID NO 5) | CAC CAA |
| HPV11 | ATC TGT GTC TAA ATC TGC TAC ATA |
| (SEQ. ID NO 6) | CAC TAA |
| HPV16 | GTC ATT ATG TGC TGC CAT ATC TAC |
| (SEQ. ID NO 7) | TTC AGA |
| HPV18 | TGC TTC TAC ACA GTC TCC TGT ACC |
| (SEQ. ID NO 8) | TGG GCA |
| HPV31 | TGT TTG TGC TGC AAT TGC AAA CAG |
| (SEQ. ID NO 9) | TGA TAC |
| HPV33 | TTT ATG CAC ACA AGT AAC TAG TGA |
| (SEQ. ID NO 10) | CAG TAC |
| HPV34 | TAC ACA ATC CAC AAG TAC AAA TGC |
| (SEQ. ID NO 11) | ACC ATA |
| HPV35 | GTC TGT GTG TTC TGC TGT GTC TTC |
| (SEQ. ID NO 12) | TAG TGA |
| HPV39 | TCT ACC TCT ATA GAG TCT TCC ATA |
| (SEQ. ID NO 13) | CCT TCT |
| HPV40 | GCT GCC ACA CAG TCC CCC ACA CCA |
| (SEQ. ID NO 14) | ACC CCA |
| HPV42 | CTG GAA CAT CTG GTG ATA CAT ATA |
| (SEQ. ID NO 15) | CAG CTG |
| HPV43 | TCT ACT GAC CCT ACT GTG CCC AGT |
| (SEQ. ID NO 16) | ACA TAT |
| HPV44 | GCC ACT ACA CAG TCC CCT CCG TCT |
| (SEQ. ID NO 17) | ACA TAT |
| HPV45 | ACA CAA AAT CCT GTG CCA AGT ACA |
| (SEQ. ID NO 18) | TAT GAC |
| HPV51 | AGC ACT GCC ACT GCT GCG GTT TCC |
| (SEQ. ID NO 19) | CCA ACA |
| HPV52 | TGC TGA GGT TAA AAA GGA AAG CAC |
| (SEQ. ID NO 20) | ATA TAA |
| HPV54 | TAC AGC ATC CAC GCA GGA TAG CTT |
| (SEQ. ID NO 21) | TAA TAA |
| HPV56 | GTA CTG CTA CAG AAC AGT TAA GTA |
| (SEQ. ID NO 22) | AAT ATG |
| HPV58 | ATT ATG CAC TGA AGT AAC TAA GGA |
| (SEQ. ID NO 23) | AGG TAC |
| HPV53 | GCA AAT TAA ACA GTA TGT TAG ACA |
| (SEQ. ID NO 24) | TGCAGA |
| HPV59 | TGT ATA CAC ACC TAC CAG TTT TAA |
| (SEQ. ID NO 25) | AGA ATG |
| HPV66 | ACT AAA TAT GAT GCC CGT GAA ATC |
| (SEQ. ID NO 26) | AAT CAA |
| HPV68 | ACC AAA TAT TTA TGA TCC TAA TAA |
| (SEQ. ID NO 27) | ATT TAA |
| HPV70 | CTG CTG TAT ATA GCC CTA CAA AGT |
| (SEQ. ID NO 28) | TTA AGG |

The present invention provides a kit for detecting and determining the genotype of HPV present in a sample which comprises: a) a tool for collection of cervical cells and reagents for DNA extraction therefrom; b) primers having the sequences depicted in SEQ. ID NO: 1-NO: 4 and reagents for amplification of the HPV DNA by a two step nested PCR method; c) an oligonucleotide chip containing probes having the sequences depicted in SEQ. ID NO: 5-NO: 28; and d) instructions for using the kit for detecting and determining the HPV genotype present in a sample.

The oligonucleotide chip for determining the genotype of the amplified HPV DNA of the present invention can be manufactured as follows: 1) synthesizing 24 types of HPV probes having the nucleotide sequences depicted in (SEQ. ID: 5-SEQ. ID: 28) which are approximately 30 nucleotides in length and have an attached amine residue on the 5′ end; and 2) immobilizing the probes in a regular array and spacing on a glass slide by a microarray method through attaching and washing. For example, DNA probes for 24 HPV subtypes with an amine (—NH2) on the 5′ end were synthesized by using a DNA synthesizer (DNA synthesizer, Polygen). After adjusting the synthesized probes to a concentration of 40 pmol/μl, they were combined with spotting buffer (3×SSC) in a ratio of 1:1. A silanated slide was divided into 8 regions, and each probe was spotted in duplicate (MicroGrid TAS, Bio-Robotics). The spotted silanated slide was dried at room temperature and then baked at 80° C. for 3 hours. It was then reacted with a blocking solution at 42° C. for one hour and reacted with isopropanol for one minute. The slide was washed 5 times for one minute each time with distilled water and then dried for use.

The procedure for performing the HPV genotype test of the present invention using an oligonucleotide chip kit is as follows:

1) cells are collected from the cervix uteri using a PAP-brush and stored in a tube containing storage buffer;

2) the cells are precipitated by centrifugation and DNA is extracted from them by cell lysis using a lysis buffer;

3) the HPV DNA is amplified by a two step nested PCR method using a primer selected from the group consisting of My09, MY11, GP+5, and Cy5-GP+6 which are specific for HPV DNA;

4) The PCR product of HPV DNA prepared in 3) is hybridized with an oligonucleotide chip containing probes that have nucleotide sequences complementary to the DNA of HPV, i.e. the 24 types listed in Table 2; and

5) The hybridized oligonucleotide chip is scanned on a scanner to determine the genotype of the HPV DNA specifically bound to the HPV probes on the chip.

The above mentioned primers used in the two step nested PCR method for amplifying HPV DNA, My09, MY11, GP+5 and Cy5-GP+6 have the sequences listed in Table 1. The 24 types of HPV probes specific to HPV DNA have the sequences listed in Table 2. The reagents, tools and equipment required for manufacturing the kit, including the oligonucleotide chips for the HPV genotype test, consist of the following:

1) a brush to collect cells from the cervix uteri (PAP-brush, Sang-A Medical, Korea);

2) proteinase K and cell lysis buffer for extracting DNA from the cells

3) 20 pmol/μl of each HPV PCR primer, My09, MY11, GP+5 and Cy5-GP+6;

10×PCR buffer;

250 units of Taq DNA polymerase (Amplitaq, PE, USA)

4) oligonucleotide chips containing 24 types of oligonucleotide probes for HPV DNA with an attached amine residue on the 5′ end;

A glass microscope slide coated with an aldehyde residue on its surface;

A chip spotter (Bio-Robotics, UK);

A hybridization chamber to divide the slide into eight regions (Multi-well hybridization chamber, Sigma, USA)

0.2% SDS solution, NaBH₄ (Sigma, USA), PBS solution, 100% ethanol;

5) 12×SSC hybridization buffer;

3N NaOH;

3N HCl;

1M Tris-HCl (pH 7.2);

10% SDS;

6) 3×SSC solution, 1×SSC solution to wash the oligonucleotide chip after hybridization; and

7) chip scanner (GSI Lumonics, USA).

Using the above-mentioned reagents, tools and equipment, the oligonucleotide chips for HPV genotype testing are manufactured and a kit containing the chip also is developed. The contents of an example kit are as follows:

1) a brush to collect cells from the cervix uteri (PAP-brush);

a tube with cell storage buffer;

2) a tube with proteinase K;

3) a tube with 20 pmol/μl of each HPV PCR primer, My09, MY11, GP+5 and Cy5-GP+6;

a tube with 10×PCR buffer;

a tube with 250 units of Taq DNA polymerase (Amplitaq, PE, USA);

4) an oligonucleotide chip with 8 well hybridization chambers (Multiwell hybridization chamber, Sigma, USA).

The above mentioned kit allows convenient extraction of DNA from cervical cells and rapid determination of the genotype of the HPVs present after amplification of the HPV DNA by a two step nested PCR method by using the oligonucleotide chip for HPV genotype testing. The required time for the test is only about 8 hours. In addition, 8 specimens can be simultaneously tested on a single HPV oligonucleotide chip. Application of the oligonucleotide chip for HPV genotype testing, as in the present invention, will make HPV genotype testing the common test method used.

The following examples will enable those skilled in the art to more clearly understand how to practice the present invention. It is to be understood that, while the invention has been described in conjunction with the preferred specific embodiments thereof, that which follows is intended to illustrate and not limit the scope of the invention. Other aspects of the invention will be apparent to those skilled in the art to which the invention pertains.

### EXAMPLE 1

### Manufacturing the DNA Chips for Determining HPV Genotypes

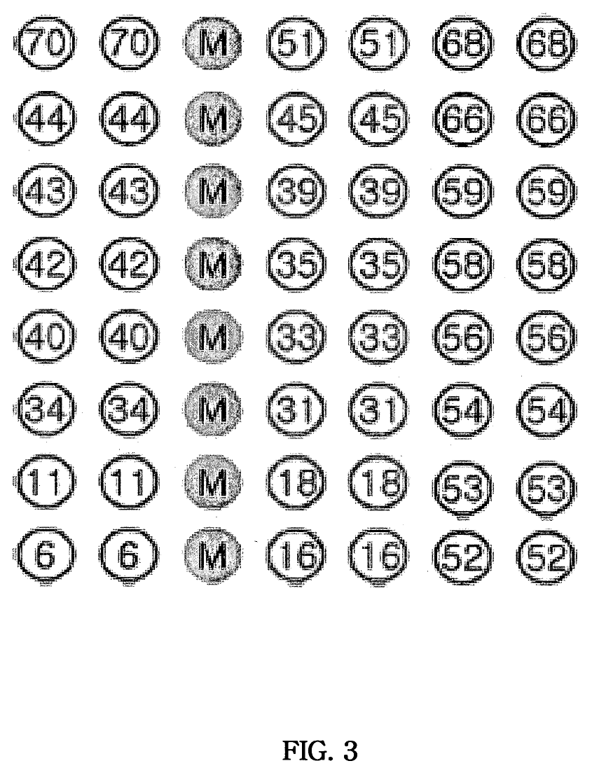

DNA probes for 24 HPV subtypes with an amine (—NH2) on the 5′ end (listed in Table 2) were synthesized by using a DNA synthesizer (DNA synthesizer, Polygen). The 5′ terminal amine groups on the probes enable the probes to bind to an aldehyde-derivatized solid surface. The DNA probes prepared were affixed to an aldehyde-derivatized surface of a solid support, i.e. a glass chip. After adjusting the synthesized probes to a concentration range of 10-99 pmol/μl, preferably 40 pmol/μl, they were combined with spotting buffer (3×SSC) in a ratio of 1:1. A silanated chip was divided into 8 regions, and each probe was spotted in an duplicate micro-array pattern (MicroGrid TAS, Bio-Robotics). Each of the low-risk group (HPV type 6, 11, 34, 40, 42, 43, 44, 70) and high-risk group (HPV types 16, 18, 31, 33, 35, 39, 45, 51, 52, 53, 54, 56, 58, 59, 66, 68) on a 384-well plate are included in the 24 types of HPV specific nucleic acid sequences. By using MicroGrid II from BioRobotics Co., 24 HPV probes and oligo bases of β-globin are spotted at a 4.4 mm spacing, parallel to each other (FIG. 3). Eight regions are spotted on a single chip. The spotted silanated chip was dried at room temperature and then baked at 80° C. for 3 hours. The chip was then reacted with a blocking solution, i.e. a mixed solution of 1.3 g NaBH₄, 375 ml of PBS and 125 ml of ethanol, at 42° C. for one hour and then reacted with isopropanol for one minute. The chip was then washed 5 times for one minute each time with distilled water. The slide was then dried and stored in a sealed container for use.

An alternative manufacturing technique of the oligonucleotide chips for HPV genotype testing is:

1) 100 pmol/μl each of 24 types of HPV probes (these sequences were listed in Table 2) were immobilized equally on each region of a microscope glass slide which had been divided into eight regions.

2) The glass slide carrying the immobilized probes was washed twice with 500 ml of 0.2% SDS and then twice with distilled water. After vigorous shaking in a mixed solution of 1.3 g NaBH₄, 375 ml of PBS and 125 ml of ethanol, the slide was washed for one minute three times with 0.2% SDS and then twice with distilled water. The slide was then dried, sealed, and stored in a sealed container for use.

### EXAMPLE 2

### Collection and Storage of Cells from the Cervix Uteri

Cervical cells were collected from the cervix uteri using a brush which is then immersed in 5 ml of PBS storage buffer in a 15 ml tube, capped and stored at 4° C. after cutting off the brush end into the tube.

### EXAMPLE 3

### DNA Extraction from the Cells

The tube of Example 2 was shaken vigorously for 2 minutes to separate the cells from the brush into the storage buffer. After precipitating the cells by centrifugation at 3000×g for 10 minutes, the supernatant was discarded. The cells were transferred to a 1.5 ml centrifuge tube using a separator (Mediland, Korea). After adding 100 μl of cell lysis buffer (10 mM Tris-HCl, 50 mM KCl, 2.5 mM MgCl₂, 0.5% Tween20, 200 μg/μl proteinase K, pH8.3), the tube was incubated at 55° C. in a water bath for 2 hours then at 95° C. for an additional 10 minutes to eliminate the activity of proteinase K.

This is a method to extract DNA from the collected cervical cells to be amplified by PCR. By this method, numerous specimens can be managed rapidly and easily. Moreover, false-positive results were not seen since cross-contamination among the specimens was prevented. In addition, the risk of environmental pollution and inconvenience were eliminated because no organic solvent is used.

Alternatively, one (1) ml of xylene was added to a specimen from which 5-10 μm paraffin sections were sampled. The specimen was reacted at 50° C. for 15 minutes, and then centrifuged at 3000 rpm for one minute. The supernatant was discarded and the procedure was repeated once. After adding 1 ml of 100% ethanol, the specimen remained at room temperature for 15 minutes, it was then centrifuged at 3000 rpm for one minute, the supernatant was discarded, and the sediment dried. 100.0 μl of DNA extracting solution was added to the sediment and mixed well. The mixture was heated at 95-100° C. for 20 minutes, allowed to remain at room temperature for 5 minutes, and then centrifuged at 14000 rpm for 5 minutes. 100 μl of separated supernatant was used for PCR, or refrigerated until use.

### EXAMPLE 4

### Amplification of HPV DNA Through a Two Step Nested PCR Method

Nested PCR was used for amplification. For the diagnosis of HPV, two primers were used: MY09/MY11 (5′-CGTCCMARRGGAWACTGATC-3′/5′-GCMCAGGGWCATAAYAATGG-3′) and GP5+/GP6+ (5′-TTTGTTACTGTGGTAGATACTAC-3/5′-Cy3-GAAAAATAAACTGTAAAT CATATTC-3′). For the control, the following β-globin primer was used: 5′-Cy3-CAACTTCATCC ACGTT CACC-3′/5′-GAAGAGCCAAGGACAGGTAC-3′.

First PCR

For pre-denaturation, a 50-μl specimen was put into a 0.6-μl PCR tube, allowed to incubate at 99° C. for 10 minutes, and then at 4° C. for 10 minutes before PCR. 50 μl of solution was made by adding 10× buffer (100 mM-KCl, 20 mM Tris-HCl (pH 8.0), 2.0 mM MgCl₂), 5 μl of 2.5 mM dNTP, 4 μl of Taq polymerase (5 units), 0.5 μl of 10 pmol MY09/MY11 primer (1.0 μl each), and 5 μl of template DNA. PCR was conducted 40 times at 95° C. for 50 seconds each time, at 55° C. for 20 seconds, and at 72° C. for 30 seconds, respectively (PCR-9700, Applied Biosystems).

Second PCR

Fifty (50) μl of solution was made by mixing the first PCR template DNA 5 μl, 5 μl of 10× buffer, 10 pmol of GP5+/GP6+ primer 1.0 μl each, 4 μl of 2.5 mM dNTP, Taq polymerase (5 units) and 1.0 μl of 10 pmol GP5+/GP6+ primer. DNA amplification was done 30 times at 94° C. for 50 seconds each time, at 55° C. for 20 seconds, and 72° C. for 20 seconds, respectively (PCR-9700, Applied Biosystems). After PCR amplification, 5.0 μl of each PCR product was electrophoresed in 2.5% agarose gel, and image analysis was performed (Multimage, BioRad).

Alternatively, HPV DNA was amplified as follows: the 5 μl of the extracted DNA of Example 2 was added to a mixture of 1×PCR buffer (50 mM KCl, 10 mM Tris-HCl pH8.4, 1.5 mM MgCl₂, 0.1% Triton X-100), 20 pmol each of MY09 and MY11, and 200 uM each of dATP, dGTP, dCTP, dTTP and 2.5 units of TaqDNA polymerase in a 200 μl PCR tube and adjusted to volume of 25 μl with distilled water. After simple vortexing and centrifugation, 35 cycles of PCR were performed on a thermal cycler (9700 thermal cycler, PE, USA) under amplification conditions as follows: 4 minutes at 95° C., followed by 35 cycles of 30 seconds each at 95° C., 30 seconds at 55° C. and 30 seconds at 72° C., followed by 4 more minutes at 72° C.

5 μl of the 1^{st} PCR product, 20 pmol each of GP+5 and Cy5-GP+6 primer, 200 uM each of dATP, dGTP, dCTP, dTTP and 2.5 units of Taq. DNA polymerase is placed in a 200-μl PCR tube, which is then adjusted to a volume of 25 μl with distilled water. After simple vortexing and centrifugation, 42 cycles of PCR were performed on a thermal cycler (9600 thermal cycler, PE, USA) under amplification conditions as follows: 4 minutes at 95° C., followed by 25 cycles of 30 seconds each at 95° C., 40 seconds at 50° C. and 30 seconds at 72° C., followed by 4 more minutes at 72° C. and then stored at 4° C. The total running time was about 2.5 hours.

### EXAMPLE 5

### Hybridization

On a silylated slide where the HPV probes were bound, a hybridization sealer attached with 8 wells was warmed at 42° C. for 5 minutes. While warming the slide, 5.0 μl of the second HPV PCR product was heated at 95° C. for 5 minutes, and cooled at 4° C. for 5 minutes. It was then remixed with 35 μl of hybridization buffer (0.3 M Mes, 10 mM MgCl₂, 0.1% SDS). A specimen of the PCR product that was mixed with the hybridization buffer was carefully pipetted into each well, making sure that no air bubbles were present. Each well was sealed with sealing tape to prevent evaporation. It was hybridized in a thermostatic oven at 42° C. for 4 hours. Non-reactive materials were removed by washing with wash buffer solution A (2×SSC, 0.1% SDS) twice, for 5 minutes each. The chip was washed again with wash buffer B (0.2×SSC) twice for 5 minutes each and finally with wash buffer solution C (0.1×SSC) once for 5 minutes. It was allowed to dry at room temperature.

Alternative hybridization process: after 8 μl of PCR product was incubated at 95° C. for 5 min, it was placed on ice for 5 min. 32 μl of 8×SSC hybridization buffer preheated at 42° C. was added. 40 μl of the above hybridization mixture from each specimen was injected carefully into each well of the above prepared slide. After sealing the injection hole of each well with cellophane tape to prevent evaporation, the slide was incubated in a thermo-stat oven at 42° C. for 2 hours. The slide was washed with 200 ml of 3×SSC wash buffer for 2 minutes and then with 200 ml of 1×SSC wash buffer while rotating at 50 rpm. The slide was carefully dried and stored in a light-resistant container.

### EXAMPLE 6

### Determination of the HPV Genotype on the Oligonucleotide Chip after Hybridization

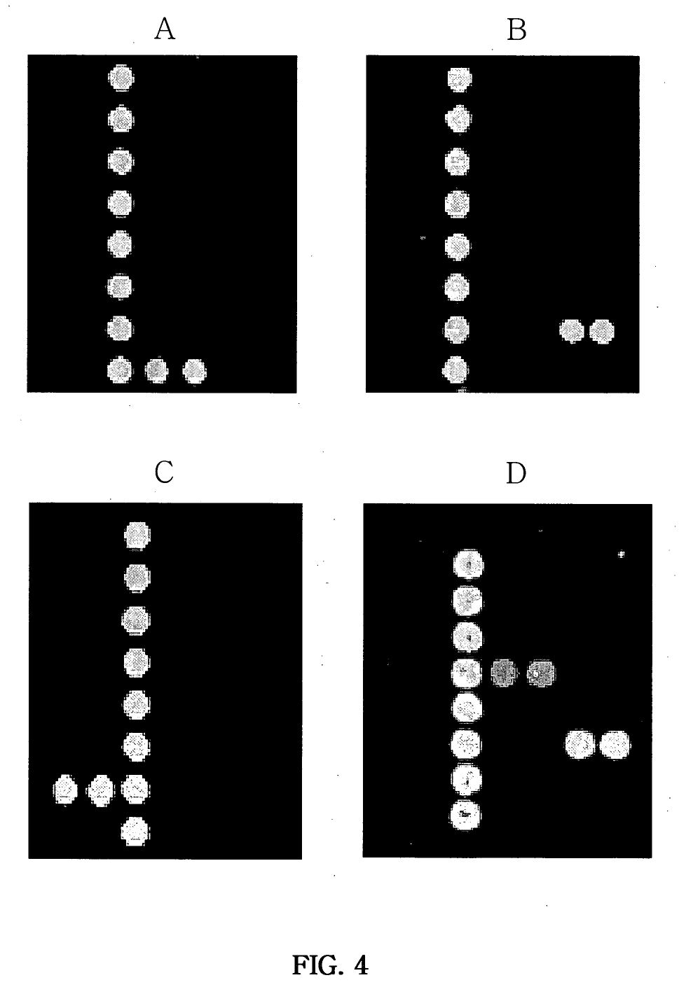

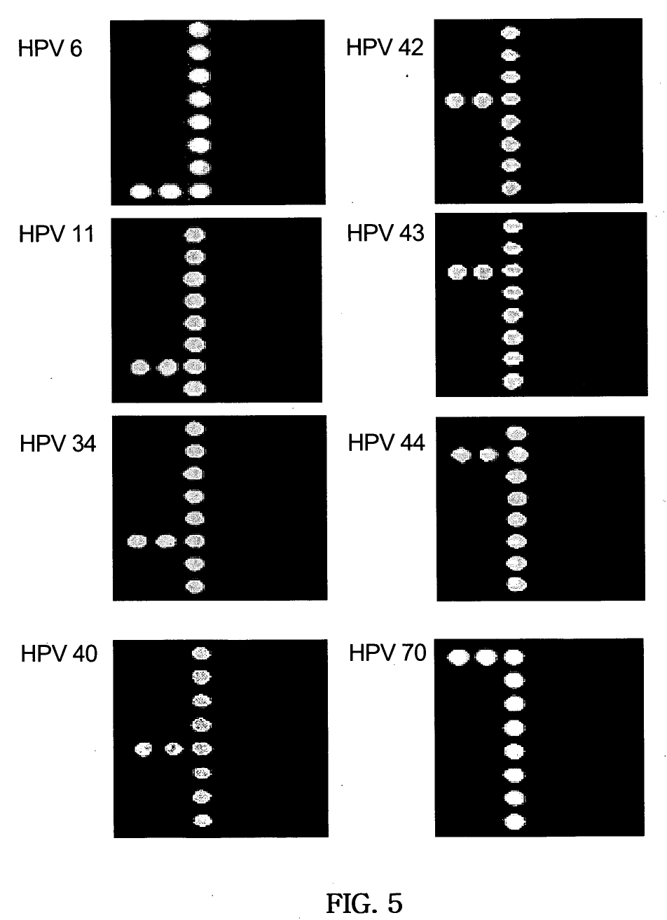

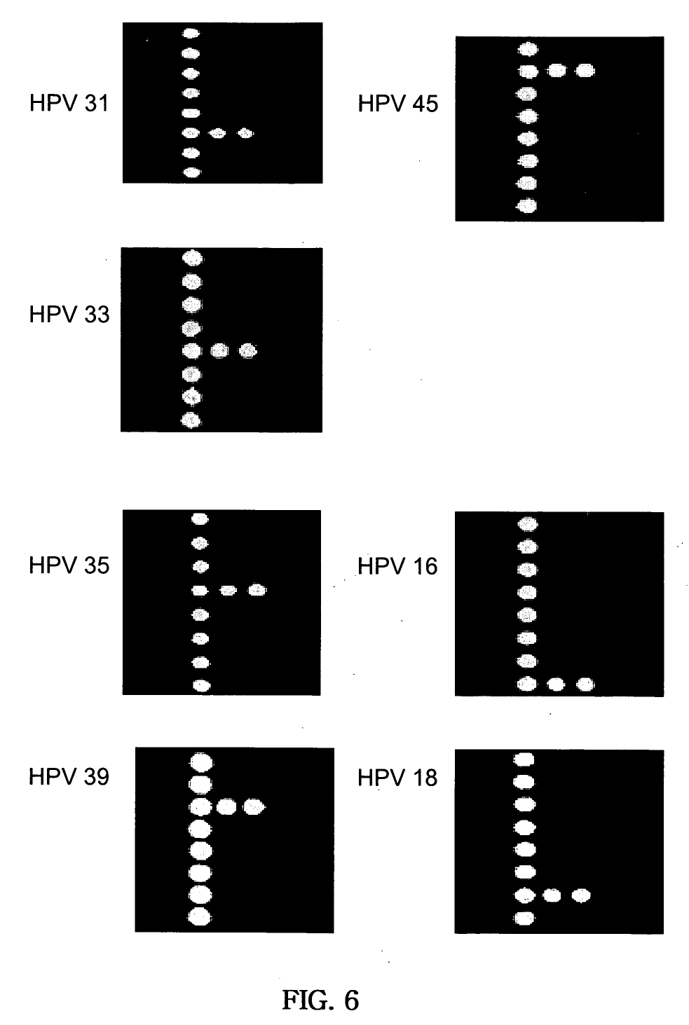

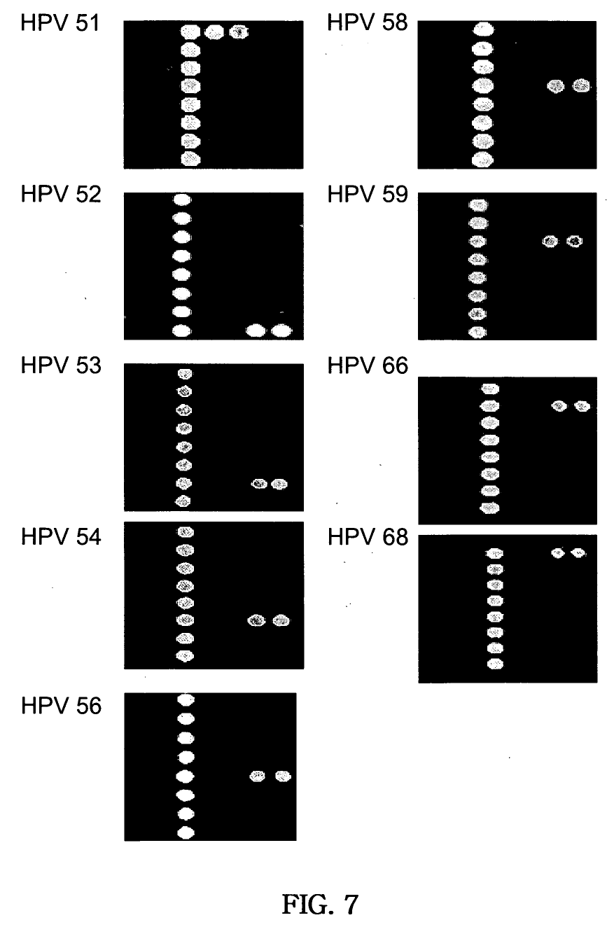

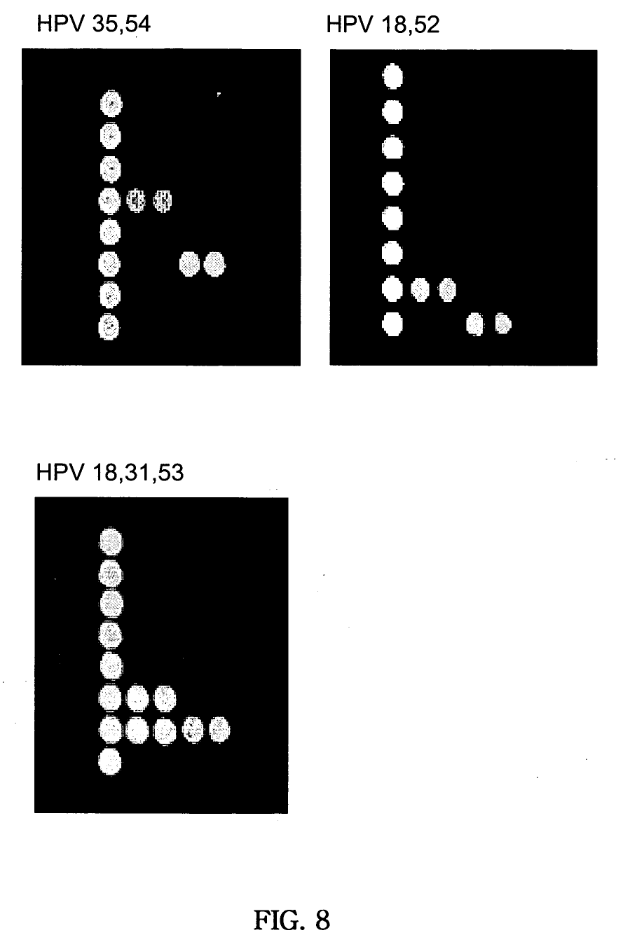

After setting the chip scanner (Scanarray4000, GSI Lumonics) to be able to detect an excitation wave length of 649 nm and an emission wave length of 670 nm, the hybridized chip in Example 5 was investigated for determining the genotype of the HPV present by detecting the loci of fluorescent signals from specific hybridization of the probe of the HPV genotype with the target HPV DNA (FIG. 4-8). The present inventive HPV chip can test eight samples at the same time and can genotype both single and multiple types HPV co-infection.

### EXAMPLE 7

### Genotype Testing of HPV in Cervical Specimens Using an Oligonucleotide Chip

149 cervical specimens which were diagnosed as showing cervical dysplasia by PAP smear testing were tested for HPV genotype using chips manufactured as in Example 1, according to the test method of Examples 2-6. The results are summarized in Table 3.

TABLE 3
HPV genotype of cervical specimens using an oligonucleotide chip for HPV
genotype testing
HPV genotype

| | | | | | | | | | | | | | | | | | | | | Multiple | | |
|---|---|---|---|---|---|---|---|---|---|---|---|---|---|---|---|---|---|---|---|---|---|---|
| | | | | | | | | | | | | | | | | | | | | infection | | |
| | | | | | | | | | | | | | | | | | | | | 2 | | |
| | | | | | | | | | | | | | | | | | | | | types | | |
| | Low-risk group | | | | | | | High-risk group | | | | | | | | | | | | or | | |
| | 6 | 11 | 34 | 40 | 42 | 43 | 44 | 16 | 18 | 31 | 33 | 35 | 39 | 45 | 51 | 52 | 54 | 56 | 58 | above | Negative | Total |
| Number | 1 | 2 | | | 4 | | 1 | 48 | 2 | 9 | 6 | 3 | 1 | 1 | 5 | 4 | 3 | 4 | 10 | 17 | 42 | 163 |
| of | | | | | | | | | | | | | | | | | | | | | | |
| specimens | | | | | | | | | | | | | | | | | | | | | | |

The above cervical specimens were collected from patients visiting the genecology department of Samsung-Jeil Hospital.

### EXAMPLE 8

### Method for HPV Genotype Testing and Procedure for Manufacture of Test Kit Using an Oligonucleotide Chip

The oligonucleotide chip kit for HPV genotype testing of the present invention comprises: 1) tools and reagents for collecting cells from the cervix uteri and extracting DNA from them; 2) reagents for amplifying the HPV DNA by PCR; and 3) oligonucleotide chips for determining the genotype of the HPV PCR product. More specifically, the kit for detection or determining the genotype of the HPV comprises:

a) tools for collecting cervical cells and reagents for DNA extraction from the cells;

b) reagents and primers for amplification of HPV DNA, which have the sequences depicted in SEQ. ID: 1, SEQ. ID: 2, SEQ. ID NO: 3 and SEQ. ID NO: 4;

c) reagents for hybridization of amplified HPV DNA with an HPV oligonucleotide chip having probes immobilized on the oligonucleotide chip having the sequences depicted in SEQ. ID:5-SEQ. ID:28; and

d) instructions on how to use the kit to detect or determine the HPV genotype in an sample.

Tools and reagents for collecting cells from the cervix uteri and extracting DNA from them according to Example 2 and Example 3, which includes a cervical cell collecting brush stored at ambient temperature; a 15 ml tube with 3 ml of PBS solution stored at 4° C.; and a 1.5 ml tube with 20 mg/ml of protease (proteinase K, Sigma, USA) stored at −20° C. This kit can manage 80 specimens of cervical cells at once and the procedure for its use is described as in Examples 3-5.

The reagents used in PCR for amplifying HPV DNA constitute the second part of the kit, comprising a 1.5 ml tube with 50 μl of each of the 4 HPV PCR primers (20 pmol/μl), My09 and MY11; a 1.5 ml tube with 50 μl of each HPV PCR primer (20 pmol/μl), GP+5 and Cy5-GP+6; a 1.5 ml tube with 10×PCR buffer; a 1.5 ml tube with 100 μl of mixed dNTP solution (10 mM, dATP, dGTP, dCTP, and dTTP); and 250 units of Taq DNA polymerase (Amplitaq, PE, USA). All contents are stored at −20° C. and the procedure for use is the same as in Examples 3-5.

The third part of the kit consists of 10 slides of oligonucleotide chips prepared according to Example 1. On each chip, 8 specimens can be tested simultaneously and the procedure for use is the same as in Examples 3-6. Therefore, using the oligonucleotide test kit for HPV genotype testing of the present invention, 80 specimens can be managed simultaneously. As shown in the results of HPV genotype testing using the oligonucleotide chip as illustrated in FIGS. 4-8, the oligonucleotide chip for HPV genotype testing of the present invention can determine the presence of 24 HPV genotypes with high specificity and detect multiple infections with HPV which are undetectable by traditional methods. In addition, it may be useful industrially since the total process time of the test, from DNA extraction to determination of the HPV genotype, is performed rapidly, within about 8 hours.

### EXAMPLE 9

### Evaluating the Accuracy of the HPV DNA Chip Test for Detection and Typing of HPV in Cervical Lesions by Comparison with the Results of HPV DNA Sequencing

In this example, the accuracy of the HPV DNA Chip test for detection and typing of HPV in cervical lesions was evaluated by comparison with results of HPV DNA sequencing. HPV DNA sequencing was performed in 282 samples, in which a positive reaction had been shown by HPV PCR, and specific HPV genotypes had been detected in the HPV DNA Chip test. The 282 study samples included 266 samples with single HPV genotypes (single infection) and 16 with multiple genotypes (multiple infections) by the HPV DNA chip test. The genotypes from HPV DNA sequencing were compared with the genotypes from the HPV DNA chip test. In the case of multiple infections, when the genotype of HPV DNA sequencing was one of the genotypes of the HPV DNA chip test, we considered that the sequencing type agreed with the genotype of HPV DNA Chip. HPV DNA sequencing was also performed in 95 HPV [Weili, check this sentence for grammar?] other genotype samples, which showed an amplified HPV-PCR product, but which were not detected by genotype-specific probes. We checked whether the sequencing genotypes of HPV-other type samples are present or not in the HPV DNA chip test.

HPV DNA Chips of the present invention were used for HPV genotyping. The HPV DNA Chip contains 24 genotype specific probes; 15 genotypes from high-risk types (HPV-16, HPV-18, HPV-31, HPV-33, HPV-35, HPV-39, HPV-45, HPV-51, HPV52, HPV-53, HPV-54, HPV-56, HPV-58, HPV-59, HPV-66, and HPV-68) and 9 genotypes of low-risk genotypes (HPV-6, HPV-11, HPV-34, HPV-40, HPV-42, HPV-43, HPV-44, HPV-54, and HPV-70).

Twenty-four type specific 30-mer oligonucleotide probes containing an amine group at the 5′ terminus were immobilized onto a chip slide glass. The slide has eight chambers, and each chamber is used for a test. Therefore, a slide tests eight samples at one time. Briefly, DNA was isolated from swab samples using a DNA isolation kit (MyGene. Co., Seoul, Korea), and target L1 regions of the HPV DNA were amplified and labeled by a single dye, indocarbocyanine-dUTP, (MEN Life Science Products, Inc., Boston, Mass.), using consensus GPd5+/Cy5-GP6d+ primers. β-Globin was amplified using PCR as the internal control. The PCR products of all samples were detected by electrophoresis through a 2.5% agarose gel, the product size of the HPV DNA was 150 base pairs (bp). 10 μl of the HPV-amplified product was denatured for 5 min at 95° C. The samples were mixed with a hybridization solution (MyGene. Co., Seoul, Korea), then applied onto the DNA chip. Hybridization was performed at 43° C. for 90 minutes and was then followed by washing with 3×SSPE for five minutes and 1×SSPE for five minutes, and then by drying at room temperature.

Hybridized HPV DNA was visualized using a DNA chip scanner (Scanarray lite; GSI Lumonics©, Ottawa, Ontario, Canada). HPV amplicons can be hybridized with corresponding type specific oligonucleotide probes and visualized on HPV DNA chip slides as double positive spots (FIG. 4) when HPV DNA is present in the amplified PCR product. As indicated in FIG. 4, the HPV-DNA chip is useful for detection of 24 HPV genotypes, all of which were amplified by PCR and subsequently hybridized to oligonucleotide probes that are specific to each genotype. The positive signal was represented as double spots. In FIG. 4, A represents a single infection of HPV-16; B indicates a single infection of HPV-53; C indicates a single infection of HPV-11 and D indicates a double infection of HPV 35 and 53.

The samples that showed a positive band at 150 bp on the gel electrophoresis but were negative on the HPV DNA chip slide were designated as HPV-other. None of the negative controls (without DNA) revealed HPV positivity.

HPV Genotyping by HPV DNA Sequencing

The primed PCR product was added to the sequencing reaction mixture. Sequencing was performed bidirectionally with the BigDye3 terminator cycle sequencing kit (PE Applied Biosystems) using an ABI PRISM 310 Genomic Analyser (PE Applied Biosystems) at a dispensing pressure of 600 mbar with 8-msec open times and 65 second cycle times. The sequencing procedure was carried out by stepwise elongation of the primer strand upon cyclic dispensation of the different deoxynucleoside triphosphates (Amersham Pharmacia Biotech). A CCD camera detected the light output resulting from nucleotide incorporation.

The results showed that in 257 (91.1%) of 282 tested samples, the genotypes of sequencing tests were in agreement with the genotypes resulting from the HPV DNA chip test of the present invention. Among 16 samples of multiple HPV genotypes coinfection, agreement was observed in nine samples. One genotype was designated in all nine samples, and the genotype from HPV DNA sequencing was one of the genotypes on the HPV DNA chip test. In the remaining seven samples of multiple HPV genotypes, genotyping was impossible due a mixed peak showing in the sequencing data. Also, genotyping was impossible in two samples of single HPV genotypes for the same reason. The results of HPV DNA sequencing designated in Table 4 indicate that the HPV-DNA chip test is an accurate method for detection and genotyping of HPV. All studied samples showed positive reaction for HPV detection. The type of HPV DNA from the chip test was in agreement with the type of the HPV DNA sequencing in 91.1% of HPV positive cervical samples.

TABLE 4
Comparison of genotype of HPV DNA sequencing and genotype by
HPV DNA chip test in 282 samples, in which specific HPV genotypes
were detected by HPV DNA chip testing.

| Agreement samples | 257 (91.1%) | |
|---|---|---|
| (multiple HPV genotypes samples) | 9 | |
| Disagreement samples | 16 (5.7%) | |
| | | The type in |
| | The type in | HPV |
| | HPV DNA chip | sequencing |
| The samples in which | 16 | 11 |
| the type in sequencing is the | 16 | 56 |
| present type in HPV DNA chip | 31 | 18 |
| (n = 7) | 52 | 54 |
| | 53 | 16 |
| | 56 | 16 |
| | 58 | 53 |
| The samples in which | 16 | 81 |
| the type in sequencing is the | 18 | 81 |
| absent type in HPV DNA chip | 18 | 84 |
| (n = 9) | 35 | 81 |
| | 53 | 61 |
| | 53 | 84 |
| | 54 | 84 |
| | 58 | 71 |
| | 70 | 74 |

| | |
|---|---|
| The samples in which genotyping is | 9 (3.2%) |
| impossible | |
| (multiple HPV genotypes samples) | 7 |

Genotyping in HPV DNA sequencing was impossible in nine cases. The nine cases consisted of seven samples of multiple genotypes and two samples of one genotype each from the HPV DNA chip test. In HPV DNA sequencing of specimens with multiple genotypes, detection of the specific type depends on the proportional dominance and number of genotypes present in the amplicon. Sometimes sequencing might not be particularly useful for identifying infection genotypes with more than one HPV genotype, because multiple infections give sequence signals from all of the available types in the specimen. Occasionally, the genotyping in HPV DNA sequencing may be possible even in multiple infections, provided one HPV type is dominant, with a low background signal from other existing genotype(s). In this study, this circumstance was noted in nine cases where one type was detected in HPV DNA sequencing, even if multiple genotypes had been found in the HPV DNA chip test.

At present, other HPV detection tests have been widely used. The southern blot hybridization test, a highly sensitive test for HPV DNA, is considered the “gold standard” for HPV detection. This test, however, is unsuitable for clinical use because it is labor intensive and requires fresh samples. The hybrid capture test is a proprietary nucleic acid hybridization signal amplification system, and a sensitive, reliable test for detecting 13 cancer-associated viral types of HPV in cervical specimens. This is a simple procedure that uses a single test to detect any type of oncogenic HPV, but it has limitations in terms of distinguishing HPV types. The polymerase chain reaction using consensus primers designed from E6 and E7 open reading frames (ORFs) followed by restriction fragment length polymorphism (RFLP) is also a highly sensitive test for the detection of HPV DNA but also has limitations in its sensitivity and genotyping capability.

In conclusion, the HPV DNA chip test can used as a diagnostic tool, since a microarray discriminates many HPV genotypes easily and also identifies multiple infections.

Furthermore, in this study, the accuracy of the HPV DNA chip test for HPV genotyping could be certified by comparison with sequencing data. In case of an equivocally abnormal smear, the information from HPV genotyping by the HPV DNA chip test could further refine the diagnosis or justify colposcopic biopsy, and will contribute to the study of cervical carcinogenesis.

It is to be understood that the above-described embodiments are only illustrative of application of the principles of the present invention. Numerous modifications and alternative embodiments can be derived without departing from the spirit and scope of the present invention and the appended claims are intended to cover such modifications and arrangements. Thus, while the present invention has been shown in the drawings and fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth in the claims.

What is claimed is:

1. A method for detection and determination of the genotype of HPV present in a biological sample, which comprises the steps of:
a) extracting DNA from the cells in a biological sample;
b) amplifying the DNA obtained in the sample by a two step nested PCR method with two sets of primers having the sequences depicted in SEQ. ID NO: 1, SEQ. ID NO: 2, SEQ. ID NO: 3 and SEQ. ID NO: 4, to give Cy5-containing amplified HPV DNA;
c) hybridizing the amplified DNA with a HPV oligonucleotide chip having multiple probes immobilized on the chip which have nucleotide sequences complementary to DNA of an HPV subtype; and
d) detecting and genotyping of HPV DNA present in the sample by detecting a Cy-5 fluorescent signal from HPV DNA bound on the chip.

2. The method according to claim 1, wherein the DNA extraction step comprises a step of protease treatment.

3. The method according to claim 1, wherein the probes are selected from the group consisting of DNA sequences depicted in SEQ. ID: 5-SEQ. ID: 28.

4. A kit for detection and determination of the genotype of HPV, comprising:
a) PCR primers for HPV genes which have the sequences depicted in SEQ. ID: 1, SEQ. ID: 2, SEQ. ID NO: 3 and SEQ. ID NO: 4;
b) an HPV oligonucleotide chip having one or more probes immobilized on the chip, which have nucleotide sequences complementary to DNA of an HPV subtype;
c) means and regents for collecting, extracting and amplifying DNA from a biological sample and hybridizing amplified DNA with the HPV oligonucleotide chip; and
c) instructions for using the kit to detect and determine the HPV genotype present in the sample.

5. The kit according to claim 4, wherein the reagents contain a protease.

6. The kit according to claim 4, wherein the probes are selected from the group consisting of DNA sequences depicted in SEQ. ID; 5-SEQ. ID: 28.

7. The kit according to claim 4, wherein the probes are immobilized on the chip in a predetermined micro-array pattern capable of detecting a single genotype or multiple genotypes of HPV simultaneously.

8. The kit according to claim 4, wherein the probes are immobilized on the chip in a predetermined micro-array pattern capable of testing multiple samples on the same chip.

9. An HPV oligonucleotide chip for detecting and genotyping HPV comprising multiple probes immobilized on a chip which have nucleotide sequences complementary to DNA of a HPV subtype, wherein the probes are immobilized on the chip in a predetermined micro-array pattern capable of detecting a single genotype or multiple genotypes of HPV simultaneously.

10. The chip according to claim 9, wherein the probes are selected from the group consisting of DNA sequences depicted in SEQ. ID; 5-SEQ. ID: 28.

11. The chip according to claim 9, wherein the probes are immobilized on the chip in a predetermined micro-array pattern capable of testing multiple samples on the same chip.

12. A process for preparing an HPV oligonucleotide chip according to claim 9, comprising the steps of:
i) preparing 5′ terminal amine-linked DNA probes, which have nucleotide sequences complementary to the DNA of a subtype of HPV;
ii) affixing the DNA probes to an aldehyde-derivatized surface of a solid support in a micro-array pattern; and
iii) drying and baking the chip, then reacting it with a blocking solution followed with isopropanol washing solution.

13. The process according to claim 12, wherein the concentration of probes ranges from 10-99 pmol/μl.

14. The process according to claim 13, wherein the concentration of probes is 40 pmol/μl.

15. The process according to claim 12, wherein the blocking solution is a mixed solution of NaBH₄, PBS and ethanol.

16. The process according to claim 12, wherein the time for the isopropanol reaction is less than one minute.


SEQUENCE LISTING (28 sequences)

SEQ ID NO: 1 - DNA, 20 bp, Artificial Sequence
  Based on HPV-L1 sequence; primer My09; m=a or c; r=g or a; w=a or t
cgtccmarrggawactgatc

SEQ ID NO: 2 - DNA, 20 bp, Artificial Sequence
  Based on HPV-L1 sequence; primer MY11; m=a or c; w=a or t; y=t or c
gcmcagggwcataayaatgg

SEQ ID NO: 3 - DNA, 23 bp, Artificial Sequence
  Based on HPV-L1 sequence; primer GP+5
tttgttactgtggtagatactac

SEQ ID NO: 4 - DNA, 25 bp, Artificial Sequence
  Based on HPV-L1 sequence; primer Cy5-GP+6
gaaaaataaactgtaaatcatattc

SEQ ID NO: 5 - DNA, 30 bp, Artificial Sequence
  probe HPV6
atccgtaactacatcttccacatacaccaa

SEQ ID NO: 6 - DNA, 30 bp, Artificial Sequence
  probe HPV11
atctgtgtctaaatctgctacatacactaa

SEQ ID NO: 7 - DNA, 30 bp, Artificial Sequence
  probe HPV16
gtcattatgtgctgccatatctacttcaga

SEQ ID NO: 8 - DNA, 30 bp, Artificial Sequence
  probe HPV18
tgcttctacacagtctcctgtacctgggca

SEQ ID NO: 9 - DNA, 30 bp, Artificial Sequence
  probe HPV31
tgtttgtgctgcaattgcaaacagtgatac

SEQ ID NO: 10 - DNA, 30 bp, Artificial Sequence
  probe HPV33
tttatgcacacaagtaactagtgacagtac

SEQ ID NO: 11 - DNA, 30 bp, Artificial Sequence
  probe HPV34
tacacaatccacaagtacaaatgcaccata

SEQ ID NO: 12 - DNA, 30 bp, Artificial Sequence
  probe HPV35
gtctgtgtgttctgctgtgtcttctagtga

SEQ ID NO: 13 - DNA, 30 bp, Artificial Sequence
  probe HPV39
tctacctctatagagtcttccataccttct

SEQ ID NO: 14 - DNA, 30 bp, Artificial Sequence
  probe HPV40
gctgccacacagtcccccacaccaacccca

SEQ ID NO: 15 - DNA, 30 bp, Artificial Sequence
  probe HPV42
ctggaacatctggtgatacatatacagctg

SEQ ID NO: 16 - DNA, 30 bp, Artificial Sequence
  probe HPV43
tctactgaccctactgtgcccagtacatat

SEQ ID NO: 17 - DNA, 30 bp, Artificial Sequence
  probe HPV44
gccactacacagtcccctccgtctacatat

SEQ ID NO: 18 - DNA, 30 bp, Artificial Sequence
  probe HPV45
acacaaaatcctgtgccaagtacatatgac

SEQ ID NO: 19 - DNA, 30 bp, Artificial Sequence
  probe HPV51
agcactgccactgctgcggtttccccaaca

SEQ ID NO: 20 - DNA, 30 bp, Artificial Sequence
  probe HPV52
tgctgaggttaaaaaggaaagcacatataa

SEQ ID NO: 21 - DNA, 30 bp, Artificial Sequence
  probe HPV54
tacagcatccacgcaggatagctttaataa

SEQ ID NO: 22 - DNA, 30 bp, Artificial Sequence
  probe HPV56
gtactgctacagaacagttaagtaaatatg

SEQ ID NO: 23 - DNA, 30 bp, Artificial Sequence
  probe HPV58
attatgcactgaagtaactaaggaaggtac

SEQ ID NO: 24 - DNA, 30 bp, Artificial Sequence
  probe HPV53
gcaaattaaacagtatgttagacatgcaga

SEQ ID NO: 25 - DNA, 30 bp, Artificial Sequence
  probe HPV59
tgtatacacacctaccagttttaaagaatg

SEQ ID NO: 26 - DNA, 30 bp, Artificial Sequence
  probe HPV66
actaaatatgatgcccgtgaaatcaatcaa

SEQ ID NO: 27 - DNA, 30 bp, Artificial Sequence
  probe HPV68
accaaatatttatgatcctaataaatttaa

SEQ ID NO: 28 - DNA, 30 bp, Artificial Sequence
  probe HPV70
ctgctgtatatagccctacaaagtttaagg